(19) United States
(12) Patent Application Publication
(10) Pub. No.: US 2026/0248720 A1
(43) Pub. Date: Aug. 27, 2026

(54) COMPOSITIONS SUITABLE FOR THE ADMINISTRATION OF CREATINE ESTERS

(71) Applicant: Ceres Brain Therapeutics, Paris (FR)

(72) Inventors: Henri BENECH, Paris (FR); Thomas JOUDINAUD, Paris (FR); Florence BENECH, Paris (FR); Aloïse MABONDZO, Paris (FR)

(21) Appl. No.: 19/162,714

(22) PCT Filed: Mar. 6, 2024
(86) PCT No.: PCT/EP2024/055927

§ 371 Date: Sep. 5, 2025

(30) Foreign Application Priority Data
Mar. 6, 2023 (EP) 23305299.2

(51) Int. Cl. A61K 9/107; A61K 31/221; A61K 47/02; A61K 47/10; A61K 47/14; A61K 47/26; A61K 47/36; A61P 3/02

(52) U.S. Cl. CPC A61K 9/107; A61K 31/221; A61K 47/02; A61K 47/10; A61K 47/14; A61K 47/26; A61K 47/36; A61P 3/02

(57) ABSTRACT

The present invention relates to a composition in the form of a particulate emulsion comprising: —at least one creatine ester or creatine ester salt, under particle form; —from 50% (w/w) to 80% (w/w) of an aqueous solvent; —from 10% (w/w) to 40% (w/w) of lipids.

### FIELD OF THE INVENTION

The present invention relates to a composition useful for the administration of creatine esters or creatine ester salts.

### TECHNICAL BACKGROUND

Creatine is an endogenous nutrient brought by the food and also produced naturally by the liver and kidneys in most vertebrates. Creatine is also known as 2-[carbamimidoyl(methyl)amino]acetic acid. It is represented by the nominal formula NH₂—C(NH)—N(CH₃)—CH₂—COOH and has the following structure:

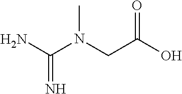

The main use of creatine is as a supplement, to increase muscle mass and enhance muscle performance.

Creatine is found mainly in muscles but also in brain, eyes, testes and kidney. In particular, creatine is essential for proper brain function, having a crucial role in energy storage and transmission, and additional roles as anti-apoptotic, anti-oxidant, neuroprotector and neuromodulator

However, when given as supplementation per os as creatine monohydrate, the brain is poorly enriched mainly due to the creatine transporter limiting transfer from the blood to the brain and with high inter-individual variability. Thus supplementing neurons with creatine remains a challenge.

Creatine deficiencies include the deficits of its biosynthesis by enzymes such as Arginine:Glycine AmidinoTransferase (AGAT) and GuanidinoAcetate MethylTransferase (GAMT), as well as deficits of its transcellular transport especially at the cerebral level. The latter, which is known as Creatine Transporter Deficiency (CTD), is a neurological disease caused by an impairment of a cellular Creatine Transporter (CrT) encoded by SLC6A8. This disorder is inherited in 70% of cases. It drastically affects brain function with mental retardation, severe speech delay, autistic-like behaviour, and seizures being the main phenotypic outcomes, although other symptoms may also be observed like failure to thrive, muscle hypotonia, neurologic and psychiatric complications, as well as cardiac and intestine manifestations. CTD is a rare disease, with about 20 000 likely cases, only a few hundred having been reported in the medical literature to date.

Different therapeutic approaches have been experimented worldwide to address the critical issue of absence of creatine in the brain cells of CTD patients. Currently, none of these approaches has led to significant benefit for CTD patients. Indeed, administration of creatine monohydrate by the oral route does not lead to any improvement as this compound cannot penetrate cerebral cells in CTD patients. Administration of creatine precursors has also failed, showing that this therapeutic attempt, although efficient for other creatine deficiency disorders (e.g. AGAT and GAMT), is not suitable to treat CTD patients.

Creatine prodrugs such as creatine fatty esters have produced promising results in animals and in vitro with patient cells. Among them, dodecyl creatine ester (DCE) could prove to be a good therapeutic option for patients suffering from CTD (Trotier-Faurion et al. (2013) J. Med. Chem. 56:5173-81; Ullio-Gamboa et al. (2019) Nanomedicine 14:1579-12593).

However, in biological fluids, such as plasma or extracellular liquids, creatine esters are rapidly degraded both by esterases and also by a very efficient non-enzymatic cyclization because of an intramolecular nucleophilic reaction leading to the inefficient creatinine. This cyclization has prevented the use of creatine esters, such as creatine ethyl ester, as an efficient supplementation in CTD.

Accordingly, an efficient delivery system is needed to overcome the rapid degradation of creatine prodrugs in plasma and in extracellular liquids in order to address CTD and other cerebral energetic deficiencies.

It has thus been proposed, in International publication WO2020/221780, to formulate creatine esters or salts thereof in a composition comprising at least one omega 3 fatty acid or a salt thereof and at least one glyceride for use in medicine, said composition being free of non-ionic surfactants.

However, the quantity of creatine esters that can be loaded and their stability in this composition are poor, the latter being due to the cyclization into inefficient creatinine, preventing this formulation to be used as a medicine. Moreover, this composition is not particularly suited for an administration by spraying, which would be the most practical form of nasal administration for creatine esters.

Accordingly, there is still a need for formulations of creatine esters in which creatine esters are stabilised and can be loaded in high quantity.

### SUMMARY OF THE INVENTION

The present invention follows from the unexpected finding by the present inventors that a particulate emulsion of lipids in water comprising dodecyl creatine ester (DCE) under particle form allowed for a high stability and for loading high quantity of DCE. Besides, the particulate emulsion is suitable for spray administration and efficiently delivered creatine or creatine esters in the brain through the nose-to-brain pathway.

Accordingly, the present invention relates to a composition in the form of a particulate emulsion comprising:
at least one creatine ester or creatine ester salt, under particle form;
from 50% (w/w) to 80% (w/w) of an aqueous solvent;
from 10% (w/w) to 40% (w/w) of lipids.

In an embodiment, the present invention relates to a composition in the form of a particulate emulsion comprising:
at least one creatine ester or creatine ester salt, under particle form;
from 50% (w/w) to 80% (w/w) of an aqueous solvent;
from 10% (w/w) to 40% (w/w) of lipids;
at least one surfactant, wherein the at least one surfactant is at a concentration of less than 15% (w/w), i.e. wherein the total of the surfactant or surfactants comprised in the composition is at a concentration of less than 15% (w/w).

Advantageously, the composition according to the invention can include more creatine ester or creatine ester salt than creatine-containing compositions for nasal administration of the prior art, i.e. the loading capacity of the composition according to the invention is increased. Moreover, the composition according to the invention allows for an increased stability of the creatine ester or creatine ester salt. Besides, the composition according to the invention is suitable for spray administration and can deliver more creatine or creatine ester to the brain through the nasal pathway than creatine-containing compositions for nasal administration of the prior art.

The present invention also relates to a method for preparing composition in the form of a particulate emulsion comprising at least one creatine ester or creatine ester salt under particle form, comprising:
Adding a solution of at least one surfactant to a first mixture of at least one creatine ester or creatine ester salt and lipids to form a second mixture,
Agitating the second mixture to form a particulate emulsion comprising the at least one creatine ester or creatine ester salt under particle form,
   wherein the at least one surfactant is at a concentration of less than 15% (w/w) in the composition in the form of a particulate emulsion.

### DETAILED DESCRIPTION OF THE INVENTION

As intended herein, the word “comprising” is synonymous to “include” or “contain”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

### Composition

Emulsions, in particular nano- or micro-emulsions, are well known to one of skill in the art and are notably described in Ullio-Gamboa et al. (2019) Nanomedicine 14:1579-1593, which is incorporated herein by reference

Preferably, the composition according to the invention does not comprise omega-3 fatty acids, such as DHA.

Preferably, the composition according to the invention further comprises at least one humectant. More preferably, the humectant is selected from the group consisting of talc, silica, vegetable stearin, magnesium stearate, glycerine, stearic acid and hyaluronate (e.g. sodium hyaluronate), in particular the humectant is glycerine and/or sodium hyaluronate. Most preferably, the composition comprises from 1% (w/w) to 10% (w/w), from 2% (w/w) to 7% (w/w), from 2.5% (w/w) to 5.5% (w/w), or from 3% (w/w) to 5% (w/w) of humectant.

Preferably, the composition according to the invention further comprises at least one stabiliser. As will be clear to the person of skill in the art, the term stabiliser relates to an emulsion stabiliser. More preferably, the stabiliser is selected from the group consisting of β-lactoglobulin, arabic gum and xanthan gum, in particular the stabiliser is xanthan gum. Most preferably, the composition comprises from 0.05% (w/w) to 1% (w/w), from 0.1% (w/w) to 0.5% (w/w), or from 0.2% (w/w) to 0.4% (w/w) of stabiliser.

Preferably, the composition according to the invention further comprises at least one surfactant, in particular at least one non-ionic surfactant. More preferably, the surfactant is selected from the group consisting of a polysorbate (e.g. polysorbate 20 or polysorbate 80), cocoglycoside, sugar esters, polyol esters, such as sorbitane esters (e.g. Span 80), lecithin, cholesterol, polyethylene glycols and derivative thereof, polyglycerols and creatine dodecyl ester, in particular the surfactant is polysorbate 80. More preferably, the composition according to the invention comprises the at least one surfactant (i.e. the total of the surfactant or surfactants comprised in the composition is) at a concentration of less than 15% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w) or 0.2% (w/w). Most preferably, the composition comprises from 0.01% (w/w) to 1% (w/w), from 0.05% (w/w) to 0.2% (w/w), or from 0.09% (w/w) to 0.11% (w/w) of surfactant.

Preferably, the composition according to the invention further comprises sodium chloride (NaCl). More preferably, the composition comprises from 0.1% (w/w) to 2% (w/w), from 0.5% (w/w) to 1.5% (w/w), or from 0.7 (w/w) to 1.0% (w/w) of NaCl.

Preferably, the aqueous solvent as defined above is water. More preferably, the water is purified water, distilled water, or deionized water. Most preferably, the composition comprises from 55% (w/w) to 75 (w/w), from 60% (w/w) to 70% (w/w), or from 64% (w/w) to 66% (w/w) of water.

“Lipids” are well-known to a person of skill in the art and refer to hydrophobic or amphiphilic small molecules which notably encompass fats, waxes, sterols, fat-soluble vitamins, fatty acids, salts and esters thereof, monoglycerides, diglycerides, triglycerides, phospholipids.

Preferably, the lipids as defined above are triglycerides, in particular medium-chain triglycerides, and/or isopropyl myristate. More preferably, the lipids are medium-chain triglycerides. More preferably also the lipids are isopropyl myristate.

Medium-chain triglycerides are well-known to the person skilled in the art. Preferably, medium-chain triglycerides are triglycerides with two or three fatty acids having an aliphatic chain, linear or branched, saturated or unsaturated, of from 6 to 12 carbon atoms. Preferably, the fatty acids are selected from the group consisting of caproic acid, caprylic acid, capric acid and lauric acid.

Preferably, the composition comprises from 15% (w/w) to 35% (w/w), from 20% (w/w) to 30% (w/w), or from 24% (w/w) to 26% (w/w) of medium-chain triglycerides and/or isopropyl myristate. More preferably, the composition comprises from 15% (w/w) to 35% (w/w), from 20% (w/w) to 30% (w/w), or from 24% (w/w) to 26% (w/w) of medium-chain triglycerides. More preferably, the composition comprises from 15% (w/w) to 35% (w/w), from 20% (w/w) to 30% (w/w), or from 24% (w/w) to 26% (w/w) of isopropyl myristate.

At least one preservative agent, such as benzalkonium chloride, may also be comprised the pharmaceutical composition as defined above.

The pharmaceutical composition according to the invention may also comprise a flavoring agent (e.g. imparting a strawberry, banana, vanilla or neroli taste), a taste masker or a taste enhancer, in particular at a concentration of from 0.001% to 0.01%, more particularly at a concentration of from 0.002% to 0.003%.

Preferably, the composition according to the invention is in the form of a particulate emulsion and comprises:
from 0.1% (w/w) to 10% (w/w) of creatine ester or creatine ester salt under particle form;
from 64% (w/w) to 66% (w/w) of water;
from 24% (w/w) to 26% (w/w) of medium-chain triglycerides.

### Creatine Ester

Preferably, the creatine ester as defined above is of the following formula (I):

(NH₂)—C(NH)—N(CH₃)—CH₂—COOR (I)

wherein R represents:
an hexosyl radical, in particular a glucosyl radical, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group;
an alkyl radical comprising from 1 to 30 carbon atoms, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group;
an alkenyl radical comprising from 2 to 30 carbon atoms, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group; or
an aryl radical comprising from 5 to 30 carbon atoms, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group.

Preferably, R represents an alkyl radical comprising from 6 to 20, more preferable from 8 to 15 carbon atoms.

Preferably, the creatine ester as defined above is selected from the group consisting of nonyl creatine ester (NCE), decyl creatine ester, undecyl creatine ester, dodecyl creatine ester (DCE), tridecyl creatine ester and tetradecyl creatine ester.

Preferably, the creatine ester as defined above is dodecyl creatine ester (DCE) and/or nonyl creatine ester (NCE). More preferably, the creatine ester as defined above is dodecyl creatine ester (DCE). More preferably, the creatine ester as defined above is nonyl creatine ester (NCE).

Preferably, the creatine ester salt is selected from the group consisting of a salt of an alkali metal, in particular sodium or potassium, a salt of an alkaline earth metal, in particular magnesium or calcium, a salt of an organic amide, more particularly of an amino acid such as arginine or lysine, a salt of an inorganic acid, such as hydrochloric acid, sulfuric acid, or hydrobromic acid, and a salt of organic acid, such as acetic acid, formic acid, fumaric acid, triflic acid, tartaric acid, oxalic acid, citric acid, trifluoroacetic acid or methanesulfonic acid.

As intended herein the creatine ester or creatine ester salt is under particle form, i.e. the creatine ester or creatine ester salt is suspended as particles in the emulsion.

Preferably, the creatine ester or creatine ester salt as defined above is micronized.

Preferably, the volume mean diameter (Dv) of the particles of the creatine ester or creatine ester salt is from about 0.1 μm to about 100 μm.

Preferably, at least 90% of the particles of the creatine ester or creatine ester salt have a volume mean diameter (Dv) equal to or more than 1 μm, 2 μm, 3 μm, 4 μm or 5 μm.

Preferably, less than 10% of the particles of the creatine ester or creatine ester salt have a volume mean diameter (Dv) equal to or less than 1 μm, 2 μm, 3 μm, 4 μm or 5 μm.

Preferably at least 90% of the particles of the creatine ester or creatine ester salt have a volume mean diameter (Dv) equal to or less than 50 μm, 30 μm or 25 μm.

Preferably, less than 10% of the particles of the creatine ester or creatine ester salt have a volume mean diameter (Dv) equal to or more than 50 μm, 30 μm or 25 μm.

Preferably, at least 50% of the particles of the creatine ester or creatine ester salt have a volume mean diameter (Dv) equal to or more than 5 μm, 6 μm, 7 μm, 8 μm, 9 μm, 10 μm, 11 μm, 12 μm, 13 μm, 14 μm or 15 μm.

Preferably, less than 50% of the particles of the creatine ester or creatine ester salt have a volume mean diameter (Dv) of equal to or less than 5 μm, 6 μm, 7 μm, 8 μm, 9 μm, 10 μm, 11 μm, 12 μm, 13 μm, 14 μm or 15 μm.

Preferably the volume mean diameter (Dv) distribution of the particles of the creatine ester or creatine ester salt is such that:
Dv(10) is of from 2 to 6 μm, preferably of from 2 to 4 μm, more preferably of from 2 to 3 μm;
Dv(50) is of from 7 to 13 μm, preferably of from 7 to 10 μm, more preferably of from 7 to 9 μm; and
Dv(90) is of from 20 to 30 μm.

Preferably, the mean width (W) of the particles of the creatine ester or creatine ester salt is from about 0.1 μm to about 100 μm.

Preferably, at least 90% of the particles of the creatine ester or creatine ester salt have a mean width (W) equal to or more than 0.5 μm, 2.5 μm or 8 μm.

Preferably, less than 10% of the particles of the creatine ester or creatine ester salt have a mean width (W) equal to or less than 0.5 μm, 2.5 μm or 8 μm.

Preferably at least 90% of the particles of the creatine ester or creatine ester salt have a mean width (W) equal to or less than 6 μm, 8 μm or 25 μm.

Preferably, less than 10% of the particles of the creatine ester or creatine ester salt have a mean width W) equal to or more than 6 μm, 8 μm or 25 μm.

Preferably, at least 50% of the particles of the creatine ester or creatine ester salt have a mean width (W) equal to or more than 2.75 μm, 5 μm or 15 μm.

Preferably, less than 50% of the particles of the creatine ester or creatine ester salt have a mean width (W) of equal to or less than 2.75 μm, 5 μm or 15 μm.

Preferably the mean width (W) distribution of the particles of the creatine ester or creatine ester salt is such that:
W(10) is of from 0.5 to 8 μm, preferably of from 2 to 7 μm, more preferably of from 0.5 to 2.5 μm;
W(50) is of from 2.75 to 15 μm, preferably of from 6 to 12 μm, more preferably of from 2.75 to 5 μm; and
W(90) is of from 6 to 25 μm, preferably of from 10 to 22 μm, more preferably of from 6 to 8 μm.

Preferably, the mean length (L) of the particles of the creatine ester or creatine ester salt is from about 0.1 μm to about 100 μm.

Preferably, at least 90% of the particles of the creatine ester or creatine ester salt have a mean length (L) equal to or more than 0.5 μm, 2.5 μm or 7 μm.

Preferably, less than 10% of the particles of the creatine ester or creatine ester salt have a mean length (L) equal to or less than 0.5 μm, 2.5 μm or 7 μm.

Preferably at least 90% of the particles of the creatine ester or creatine ester salt have a mean length (L) equal to or less than 18 μm, 22 μm or 40 μm.

Preferably, less than 10% of the particles of the creatine ester or creatine ester salt have a mean length (L) equal to or more than 18 μm, 22 μm or 40 μm.

Preferably, at least 50% of the particles of the creatine ester or creatine ester salt have a mean length (L) equal to or more than 5 μm, 9 μm or 20 μm.

Preferably, less than 50% of the particles of the creatine ester or creatine ester salt have a mean length (L) of equal to or less than 5 μm, 9 μm or 20 μm.

Preferably the mean length (L) distribution of the particles of the creatine ester or creatine ester salt is such that:
L(10) is of from 0.5 to 7 μm, preferably of from 0.5 to 2.5 μm;
L(50) is of from 5 to 20 μm, preferably of from 5 to 9 μm; and
L(90) is of from 18 to 40 μm, preferably of from 18 to 22 μm.

Methods for determining the volume mean diameter (Dv), the mean width (W), the mean length (L), and the Dv, W and L distribution of particles are well known to the person skilled in the art and notably involve the microscopy technology, in particular by means of a Malvern Morphologi 4 or Morphology G3S apparatus, or the laser diffraction technology, in particular by means of a Malvern Mastersizer 3000 Hydro MV apparatus.

Preferably, the composition according to the invention comprises from 0.1% (w/w) to 10% (w/w), from 1% (w/w) to 8% (w/w) or from 2% (w/w) to 6% (w/w) of the creatine ester or creatine ester salt.

Preferably, the composition according to the invention comprises:
From 3 to 5% (w/w) Micronized DCE, HCl salt;
From 60 to 70% (w/w) purified water;
From 0.5 to 1.0% (w/w) sodium chloride;
From 1.0 to 6.0% (w/w) glycerin (4811);
From 0.1 to 0.3% (w/w) sodium hyaluronate;
From 20 to 30% (w/w) Triglyceride based lipids (Kollisolv® MCT70, BASF);
From 0.1 to 0.5% (w/w) xanthan gum;
From 0.05 to 0.20% (w/w) polysorbate80,
Optionally from 0.01 to 0.05% (w/w) Neroli.

### Use in Therapy

Preferably, the composition according to the invention is a pharmaceutical composition or a medical device

Preferably, the pharmaceutical composition or medical device as defined above further comprises at least one pharmaceutically acceptable vehicle, carrier or excipient, in particular suitable for an administration through the topical or the nasal route.

Preferably, the composition according to the invention is suitable for spray administration. The sprayer may be single-dose, bi-dose or multidose device.

Preferably, the composition according to the invention is a dermatological composition.

Preferably, the composition, in particular the dermatological composition, according to the invention is suitable for topical administration, in particular by a spray, a cream, an ointment, a gel, or a balm.

Preferably, the composition according to the invention is administered at a unit dose of from 50 μL to 2 mL.

Preferably, the composition according to the invention is for use as a medicament.

Preferably, the dermatological composition according to the invention is for use as a dermatological medicament.

Preferably, the composition according to the invention is for use in the prevention or treatment of a creatine deficiency, such as the creatine transporter deficiency (CTD) syndrome, GAMT deficiency, or AGAT deficiency, a mitochondrial disease, such as Leich Syndrome, Leber Hereditary Optic Neuropathy or MELAS syndrome, a neurodegenerative disease, in particular Parkinson's disease and related diseases, or multiple system atrophy (MSA), a neuromuscular disorder, hypoxia, an ischemic brain disease, such as stroke, an cardiovascular disease, a muscular dystrophy, a skin disorder, such as atopic dermatitis or skin injury, amyolateral sclerosis, adrenoleukodystrophy, leukodystrophy, Rett syndrome, Kabuki syndrome, or inflammation.

More preferably, the composition according to the invention is for use in the prevention or treatment of a creatine transporter deficiency (CTD).

### Use as a Cosmetic

Preferably, the composition according to the invention is a cosmetic composition.

The present invention also relates to the use of the composition according to the invention as a cosmetic or for cosmetic applications.

When used as a cosmetic composition the composition according to the invention is preferably applied topically on the skin of an individual, more preferably a human individual, in particular by a spray, a cream, an ointment, a gel, or a balm. The cosmetic use of the composition according to the invention is intended to improve the appearance of the skin on which it is applied. In that case the individual preferably does not suffer from a disease, in particular a skin disease, that can be treated by the composition according to the invention.

### Individual

Preferably, the composition according to the invention is to be administered to an individual which is a mammal, in particular a human.

### Method

Preferably, the solution comprising at least one surfactant further comprises NaCl and/or at least one humectant. The solvent of the solution is an aqueous solvent, preferably water.

Preferably, a solution of at least one stabiliser is mixed with the particulate emulsion.

The invention will be further described by the following non-limiting figures and examples.

### DESCRIPTION OF THE FIGURES

FIG. 1 represents the plume (spray) angle measures in degree (vertical axis) for 4 tested pumps with the composition in the form of a particulate emulsion according to the invention (RT: room temperature)

FIG. 2 represents the ovality of the spray (vertical axis) measured for the 4 tested pumps with the composition in the form of particulate emulsion according to the invention (RT: room temperature).

FIG. 3 represents de intra-brain concentration of DCE (vertical axis, in ng/g) in SLC6A8 KO mice administered intra-nasally with a vehicle (KO-Vehicle), a non-particulate DCE formulation (KO-4.4s), or a DCE composition in the form of a particulate emulsion according to the invention (KO-510).

FIG. 4 represents de intra-brain concentration of creatine (vertical axis, in ng/g) in SLC6A8 KO mice administered intra-nasally with a vehicle (KO-Vehicle), a non-particulate DCE formulation (KO-4.4s), or a DCE composition in the form of a particulate emulsion according to the invention (KO-510).

FIG. 5 represents a microscopic view of a composition in the form a particulate emulsion according to the invention (up, polarized) with a DCE concentration of 40 mg/g.

### EXAMPLE

### 1. Preparation of a Particulate Emulsion of Dodecyl Creatine Ester (DCE)

A composition with the following components is prepared:

TABLE 1
Composition according to the invention (for 100 g)

| Component | % (w/w) | Function |
|---|---|---|
| Micronized DCE, HCl salt | 3 to 5 | API |
| Purified Water | 60 to 70 | Aqueous phase |
| Sodium Chloride | 0.5 to 1.0 | Osmotic agent |
| Glycerin (4811) | 1.0 to 6.0 | Humectant |
| Optionally sodium hyaluronate | 0.1 to 0.3 | Humectant |
| Triglyceride based lipids | 20 to 30 | Oil phase |
| (Kollisolv ® MCT70, BASF) | | |
| Xanthan gum | 0.1 to 0.5 | Stabiliser |
| Polysorbate80 | 0.05 to 0.20 | Surfactant |
| Optionnally Neroli | 0.01 to 0.05 | Taste masker |

Briefly, a first mixture (Mixture 1) is prepared as follows:

In a beaker, dissolve NaCl in water. Add glycerol and polysorbate80 and use a magnetic stir bar until complete dissolution to obtain a homogenous mixture.

A second mixture (Mixture 2) is made as follows:

In a beaker dissolve the DCE hydrochloride salt in the medium-chain triglycerides. Use a magnetic stir bar until complete dissolution to obtain a homogenous mixture.

Mixture 1 is mixed under agitation (magnetic stir bar) with Mixture 2 to obtain a third mixture (Mixture 3) under agitation as follows:

Set up an Ultraturax in the beaker containing the aqueous phase (Mixture 1) and gently pour the oily phase (Mixture 2) under agitation with Ultraturax. Mix until obtention of a white emulsified phase. Then progressively lower the speed of the Ultraturax until stop.

A fourth mixture (Mixture 4) is made as follows:

In a beaker dissolve xanthan gum in of water with a magnetic stir bar until complete dissolution.

Finally, Mixture 3 is mixed with Mixture 4 to obtain a homogenous emulsion as follows: Gently pour Mixture 4 into Mixture 3 under Ultraturax agitation. Add the taste masker. Mix until complete and homogenous emulsion. Leave it to stand and perform a microscopic control to check the emulsion homogeneity (FIG. 5).

Alternatively, a first mixture (Mixture 1) is prepared as follows:

In a beaker, dissolve NaCl in water. Then dissolve xanthan gum in the water phase with a magnetic stir bar until complete dissolution. Add glycerol and polysorbate80 and use a magnetic stir bar until complete dissolution to obtain a homogenous mixture.

A second mixture (Mixture 2) is made as follows:

In a beaker dissolve the DCE hydrochloride salt in the medium-chain triglycerides. Use a magnetic stir bar until complete dissolution to obtain a homogenous mixture.

Mixture 1 is mixed under agitation (magnetic stir bar) with Mixture 2 to obtain a third mixture (Mixture 3) under agitation as follows:

Set up an Ultraturax in the beaker containing the aqueous phase (Mixture 1) and gently incorporate the oily phase (Mixture 2) under agitation with Ultraturax. Mix until obtention of a homogenous white particulate emulsion. Then progressively lower the speed of the Ultraturax until stop. Add the taste masker. Mix until complete and homogenous emulsion. Leave it to stand and perform a microscopic control to check the particulate emulsion homogeneity (FIG. 5).

### 2. Spray Administration of the Composition

The composition was sprayed by means of several pumps as follows:

| Pumps | Manufacturer | Delivered volume |
|---|---|---|
| CPS | APTAR | 100 μL |
| 3K | URSATEC | 100 μL |
| PFP N | SILGAN | 140 μL |
| VP7 CB18 | APTAR | 100 μL |

Sprayability was assessed by the calculation of plume (spray) angle, plume (spray) ovality, and homogeneity of the delivery.

### 2.1. Spray Angle

The spray angle is determined from the tangent of the spray cone at 60 mm from the spay nozzle tip.

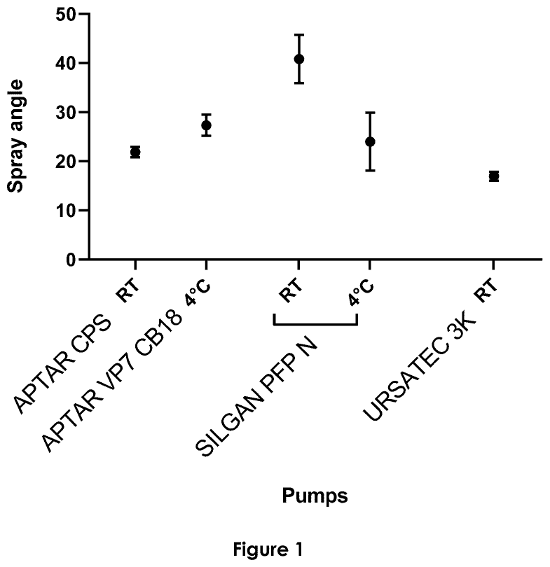

The results of the plume (spray) angle measurements (in degrees) for the 4 pumps with the particulate emulsion are shown in FIG. 1 and in the following Table 1:

| | Pumps | | | | |
|---|---|---|---|---|---|
| | APTAR | APTAR | | | URSATEC |
| | CPS | VP7 CB18 | SILGAN PFP N | | 3K |
| | Temperature | | | | |
| | RT | 4° C. | RT | 4° C. | RT |
| Plume angle | 21.9 | 27.3 | 40.8 | 24.0 | 17.0 |
| SD | 1.05 | 2.1 | 4.9 | 5.9 | 0.9 |
| CV (%) | 4.8 | 7.7 | 12.0 | 24.6 | 5.3 |

RT = room temperature

A large spray angle is sought. In the present example, the particulate emulsion for spray allows obtaining sprays with a large angle for all pumps, with the pump PFP N from SILGAN exhibiting the larger spray angle, the VP7 CB18 pump from APTAR being also efficient for spraying the particulate emulsion.

### 2.2. Spray Ovality

The spray ovality is obtained from the spray pattern calculated at 6 cm from the tip of the pump. Spray ovality is calculated with the formula: Dmax/Dmin.

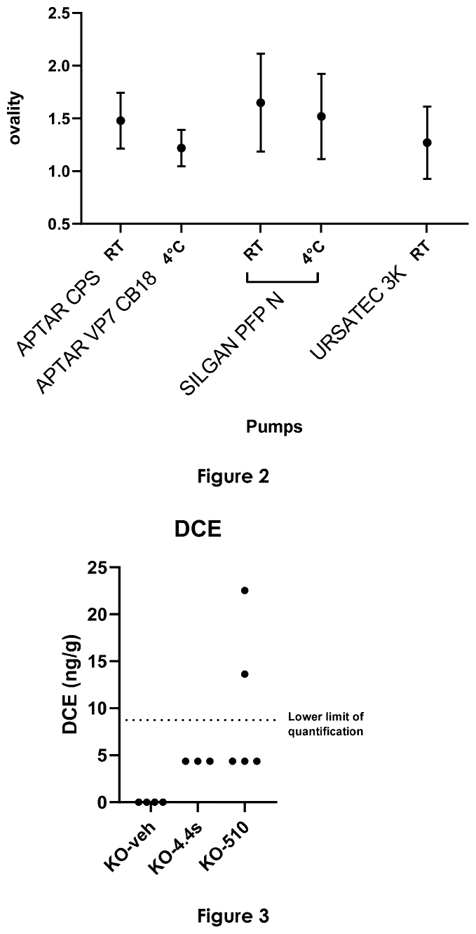

The results of the measurement of the spray ovality for the 4 pumps with the particulate emulsion for spray at different temperatures are shown in FIG. 2 and in the following

TABLE 2

| | Pumps | | | | |
|---|---|---|---|---|---|
| | APTAR | APTAR | | | URSATEC |
| | CPS | VP7 CB18 | SILGAN PFP N | | 3K |
| | Temperature | | | | |
| | RT | 4° C. | RT | 4° C. | RT |
| Ovality | 1.5 | 1.2 | 1.7 | 1.5 | 1.3 |
| SD | 0.26 | 0.17 | 0.46 | 0.4 | 0.34 |
| CV (%) | 18 | 14 | 28 | 26 | 27 |

An ovality close to 1 is sought. In the present example, spray ovalities of good quality are obtained with all the tested pumps, with a slight advantage of the pump APTAR VP7-CB18.

### 2.3. Homogeneity of the Delivery

### Homogeneity of the Delivered Dose to Entirely Empty the Pump

Delivered doses were weighted during a pump life cycle. Each set of experiment was performed by two different investigators to consider a potential inter-individual variability. Some experiments were performed either at room temperature or after storage of the device at 4° C. for at least 24 hours.

The CPS (APTAR), VP7 CB18 (APTAR) and the 3K (URSATEC) delivered consistent doses during the first 70 actuations.

### Homogeneity of the Delivered Dose According to the European Pharmacopeia

The homogeneity of the DCE dose delivered was measured for the association of the particulate emulsion with 3 pumps: APTAR CPS, APTAR VP7 CB18 and APTAR CPS. This test was performed according to the European pharmacopeia for nasal preparation.

The DCE content was quantified in the delivered formulation of 10 pump actuations (3 at the beginning, 4 in the middle and 3 at the end of pump life). The results are presented in the Tables 3-5 hereafter:

TABLE 3
homogeneity of the DCE doses delivered according to the European
pharmacopeia with the APTAR CPS pump (values in bold are in the
accepted range, value in italic are outside the accepted range)

| | # of | Quantity | DCE | | | % of the | % of |
|---|---|---|---|---|---|---|---|
| | actu- | delivered | content | Mean | CV | target | the |
| Pump | ation | (mg) | (mg) | (mg) | (%) | (4 mg) | mean |
| APTAR | 1 | 91.5 | 3.75 | 3.93 | 14.7 | 93.79 | 95.46 |
| CPS | 2 | 91.8 | 4.14 | | | 103.52 | 105.36 |
| | 3 | 97.7 | 4.42 | | | 110.38 | 112.34 |
| | 4 | 83.0 | 4.19 | | | 104.66 | 106.52 |
| | 5 | 87.5 | 3.87 | | | 96.65 | 98.37 |
| | 6 | 94.0 | 4.34 | | | 108.38 | 110.30 |
| | 7 | 97.8 | 4.70 | | | 117.53 | 119.62 |
| | 8 | 60.3 | 2.72 | | | 68.06 | 69.27 |
| | 9 | 74.3 | 3.31 | | | 82.64 | 84.11 |
| | 10 | 81.3 | 3.88 | | | 96.94 | 98.66 |

| | |
|---|---|
| % of the target | 98.3 |
| (4 mg) | |

TABLE 4
homogeneity of the DCE doses delivered according
to the European pharmacopeia with the APTAR VP7
CB18 pump (values in bold are in the accepted range,
value in italic are outside the accepted range)

| | | | | | | % of | |
|---|---|---|---|---|---|---|---|
| | # of | Quantity | DCE | | | the | % of |
| | actu- | delivered | content | Mean | CV | target | the |
| Pump | ation | (mg) | (mg) | (mg) | (%) | (4 mg) | mean |
| APTAR | 1 | 107.8 | 3.94 | 4.17 | 10.1 | 98.41 | 94.42 |
| VP7 | 2 | 107.2 | 4.46 | | | 111.55 | 107.03 |
| CB18 | 3 | 109.6 | 4.34 | | | 108.49 | 104.10 |
| | 4 | 108.0 | 4.21 | | | 105.22 | 100.96 |
| | 5 | 106.3 | 4.29 | | | 107.19 | 102.85 |
| | 6 | 106.4 | 4.84 | | | 120.98 | 116.08 |
| | 7 | 106.7 | 4.51 | | | 112.80 | 108.24 |
| | 8 | 87.2 | 3.34 | | | 83.45 | 80.07 |
| | 9 | 107.4 | 3.91 | | | 97.83 | 93.87 |
| | 10 | 105.9 | 3.85 | | | 96.28 | 92.38 |

| | |
|---|---|
| % of the target | 104.2 |
| (4 mg) | |

TABLE 5
homogeneity of the DCE doses delivered according to the European
pharmacopeia with the APTAR CPS pump (values in bold are in the
accepted range, value in italic are outside the accepted range)

| | | | | | | % of | |
|---|---|---|---|---|---|---|---|
| | # of | Quantity | DCE | | | the | % of |
| | actu- | delivered | content | Mean | CV | target | the |
| Pump | ation | (mg) | (mg) | (mg) | (%) | (3 mg) | mean |
| APTAR | 1 | 83.7 | 2.50 | 3.13 | 13.7 | 83.21 | 79.85 |
| CPS | 2 | 94.4 | 3.01 | | | 100.44 | 96.39 |
| | 3 | 96.9 | 3.39 | | | 112.88 | 108.32 |
| | 4 | 102.4 | 3.38 | | | 112.83 | 108.27 |
| | 5 | 101.6 | 3.71 | | | 123.80 | 118.80 |
| | 6 | 102.7 | 3.49 | | | 116.24 | 111.55 |
| | 7 | 102.1 | 3.48 | | | 116.03 | 111.34 |
| | 8 | 80.0 | 2.70 | | | 89.94 | 86.30 |
| | 9 | 82.0 | 2.56 | | | 85.30 | 81.86 |
| | 10 | 91.7 | 3.04 | | | 101.41 | 97.32 |

| | |
|---|---|
| % of the target | 104.2 |
| (3 mg) | |

Acceptation criteria in the European pharmacopeia for nasal preparation are:
A mean of the delivered dose between 85 and 115% of the target dose;
A maximum of one actuation outside the range 75%-125% of the target and none outside the range 65-135%.

For the 3 combinations pump/particulate emulsion tested, all the criteria are fulfilled.

### 4. Stability of DCE in the Particulate Emulsion

The chemical stability of DCE in the particulate emulsion was monitored using an HPLC/UV method at 4° C. and at room temperature. The results are shown in Tables 6-7 below.

TABLE 6
Stability of DCE (% of initial content) in two
batches of the particulate emulsion stored at
4° C. and estimations at 18 months of storage.

| | | Batch 1 | Batch 2 |
|---|---|---|---|
| Interval of analysis (days) | 7-20 | 100% | 100% |
| | 30-115 | 100% | 99.3% |
| | over 115 | 98.9% | |

| | | |
|---|---|---|
| Decay slope | −0.003 | −0.006 |
| Estimation of remaining content after | 98.1% | 96.8% |
| 18 months of storage at 4° C. | | |

DCE is stable over more than 3 months upon storage in the fridge (4° C.) and an extrapolation with the slope of the decay indicate that more than 95% should be remaining over 18 months.

TABLE 7
Stability of DCE (% of initial content) in two batches
of the particulate emulsion stored at room temperature
(20-25° C.) and estimations at 1 month of storage

| | | Particulate | Particulate |
|---|---|---|---|
| | | emulsion | emulsion |
| | | Batch 1 | Batch 2 |
| Interval of analysis in | 7-20 | 99% | 99.6% |
| days | 30-115 | 95.9% | 94.0% |
| | over 115 | 91.7% | |

| | | |
|---|---|---|
| Decay slope | −0.03 | −0.06 |
| Calculation of remaining content (%) after | 99% | 98% |
| 1 month of storage at room temperature | | |

DCE is stable over more than 20 days upon storage at room temperature (20-25° C.) and an extrapolation with the slope of the decay indicate that 99% is remaining over 1 month.

In view of the foregoing the particulate emulsion according to the invention is suitable for a long-term storage in the fridge. The good stability at room temperature for a short period allows the particulate emulsion to be stored out of the fridge for a period of 1 month at least.

### 5. Efficacy of Delivery of Creatine in the Brain

Three groups of SLC6A8 knock-out (KO) mice were respectively administered by the intranasal route a vehicle (the same emulsion without DCE), a DCE emulsion in which the formation of DCE particles is limited by a high concentration of surfactant (Polysorbate 80) also promoting their dissolution (KO-4.4s) and a composition in the form of particulate emulsion of DCE according to the invention (KO-510).

| Component | % (w/w) |
|---|---|
| Dodecyl Creatine Ester | 4 |
| (DCE) | |
| Triglycerides of caprylic | 19 |
| and capric acids | |
| (Kollisolv MCT, BASF) | |
| Polysorbate 80 | 22.3 |
| NaCl 0.9% | 54.7 |

Briefly, formulations KO-4.4s and KO-510 contained 40 and 38 mg/ml of DCE, respectively and 10 μL of the formulations were delivered to each nostril once a day for 1 month.

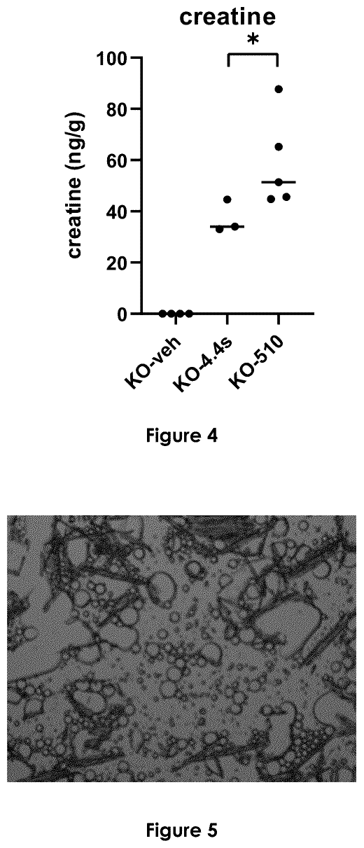

DCE and its active metabolite creatine were then detected by LC/MS/MS in one brain hemisphere of each mice. The results are reported in the FIGS. 3-4.

DCE and creatine intra-brain concentrations are higher in the mice group administered with the particulate emulsion for spray according to the invention (i.e. KO-510). This establishes that the composition in the form of a particulate emulsion according to the invention is more efficient to deliver DCE and its active metabolite creatine to the brain, in particular via the nasal pathway by spraying.

Besides, safety studies conducted in animals show that while the particulate emulsion according to the invention administered through the nasal route is safe, i.e. well tolerated by the nasal mucosa (rats at 6 mg/kg/day and dogs at 1 mg/kg/day, both for 30 days by the nasal route), formulation KO-4.4s induces local toxicity in nasal cavities (cynomolgus at from 0.9 to 2.3 mg/kg/day for 30 days), as histopathological studies show cell inflammation, necrosis and presence of cell debris and/or exudate.

1. A composition in the form a particulate emulsion comprising:
at least one creatine ester or creatine ester salt, under particle form;
from 50% (w/w) to 80% (w/w) of an aqueous solvent; and
from 10% (w/w) to 40% (w/w) of lipids.

2. The composition according to claim 1, wherein the composition does not comprise omega-3 fatty acids.

3. The composition according to claim 1, further comprising at least one humectant.

4. The composition according to claim 1, further comprising at least one stabiliser.

5. The composition according to claim 1, further comprising at least one surfactant, wherein the at least one surfactant is at a concentration of less than 15% (w/w).

6. The composition according to claim 1, further comprising sodium chloride (NaCl).

7. The composition according to claim 1, wherein the aqueous solvent is water.

8. The composition according to claim 1, wherein the lipids comprise, or consist of, triglycerides.

9. The composition according to claim 1, wherein the creatine ester is of the following formula (I):
NH₂—C(NH)—N(CH₃)—CH₂—COOR (I)
wherein R represents:
an hexosyl radical, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group;
an alkyl radical comprising from 1 to 30 carbon atoms, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group;
an alkenyl radical comprising from 2 to 30 carbon atoms, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group; or
an aryl radical comprising from 5 to 30 carbon atoms, optionally substituted by at least one group selected from the list consisting of an alkyl group comprising from 1 to 5 carbon atoms, an aryl group comprising from 5 to 10 carbon atoms, an alkoxy group comprising from 1 to 5 carbon atoms, an halogen atom, a cyano group, a trifluoromethyl group or a nitro group.

10. The composition according to claim 1, wherein the creatine ester is dodecyl creatine ester (DCE) and/or nonyl creatine ester (NCE).

11. The composition according to claim 1, wherein the creatine ester or creatine ester salt is micronized.

12. The composition according to claim 1, comprising from 0.1% (w/w) to 10% (w/w) of the creatine ester or creatine ester salt.

13. The composition according to claim 1, which is suitable for spray administration.

14. The composition according to claim 1, which is a pharmaceutical composition or a medical device.

15. (canceled)

16. A method for prevention or treatment of a creatine deficiency; a mitochondrial disease, such as Leich Syndrome, Leber Hereditary Optic Neuropathy or MELAS syndrome; a neurodegenerative disease, in particular Parkinson's disease and related diseases, or multiple system atrophy (MSA); a neuromuscular disorder; hypoxia; an ischemic brain disease, such as stroke; a cardiovascular disease; a muscular dystrophy; a skin disorder, such as atopic dermatitis or skin injury; amyolateral sclerosis; adrenoleukodystrophy; leukodystrophy; Rett syndrome; Kabuki syndrome; or inflammation, comprising administering the composition according to claim 1 to a human.

17. The method according to claim 16, for prevention or treatment of a creatine transporter deficiency (CTD).

18. A method for preparing a composition in the form of a particulate emulsion comprising at least one creatine ester or creatine ester salt under particle form, comprising:
adding a solution of at least one surfactant to a first mixture of at least one creatine ester or creatine ester salt and lipids to form a second mixture; and
agitating the second mixture to form a particulate emulsion comprising the at least one creatine ester or creatine ester salt under particle form,
wherein the at least one surfactant is at a concentration of less than 15% (w/w) in the composition in the form of a particulate emulsion.

19. The composition according to claim 5, wherein the at least one surfactant is at least one non-ionic surfactant.

20. The composition according to claim 8, wherein the triglycerides are medium-chain triglycerides.
